(19) United States
(12) Patent Application Publication
(10) Pub. No.: US 2025/0034413 A1
(43) Pub. Date: Jan. 30, 2025

(54) METHODS FOR DECELLULARIZING ANIMAL TISSUE AND BIOINKS DERIVED THEREFROM

(71) Applicant: VIRGINIA COMMONWEALTH UNIVERSITY, Richmond, VA (US)

(72) Inventors: Lucas C. OLSON, Montpelier, VA (US); Michael J. MCCLURE, Richmond, VA (US); Zvi SCHWARTZ, Richmond, VA (US); Tri M. NGUYEN, Richmond, VA (US); Barbara D. BOYAN, Richmond, VA (US)

(21) Appl. No.: 18/716,703

(22) PCT Filed: Dec. 6, 2022
(86) PCT No.: PCT/US2022/051897

§ 371 Date: Jun. 5, 2024

Related U.S. Application Data
(60) Provisional application No. 63/287,316, filed on Dec. 8, 2021.

(51) Int. Cl. C09D 11/02; C12N 5/077; C12N 9/64

(52) U.S. Cl. CPC C09D 11/02; C12N 5/0658; C12N 9/6413; C12Y 304/23001

(57) ABSTRACT

A method of decellularizing in an animal tissue includes digesting the animal tissue; treating the animal tissue with a surfactant: treating the animal tissue with at least one zwitterionic detergent to form a decellularized animal tissue; and treating the decellularized animal tissue with at least one advanced glycation end-product (AGE) inhibitor to reduce AGE crosslinking in the decellularized animal tissue. The decellularized animal tissue may be ground into a powder and dissolved in a digest solution to form a bioink composition useful for 3D printing an in vitro model of healthy or diseased tissue.

### FIELD OF THE INVENTION

The invention is generally related to methods of decellularizing animal tissue which include treatment with zwitterionic detergents followed by treatment with an advanced glycation end-product (AGE) inhibitor to reduce AGE crosslinking in the decellularized tissue. Bioinks formed from the decellularized tissue may be used to 3D-print in vitro models of healthy or diseased tissue.

### BACKGROUND OF THE INVENTION

Musculoskeletal injury increases due to falls and other accidental injuries in old age, and age-dependent alterations to skeletal muscle structure and function are the primary causal factors of such incidents. The etiology of muscle atrophy due to aging, or sarcopenia, includes loss in muscle strength, muscle fiber wasting, increased intramuscular connective tissue, and a disruption of the muscle stem cell population that results in muscle that is weaker, stiffer, and less able to regenerate. Muscular aging is multifactorial, involving extrinsic and intrinsic mechanisms that attack both the cellular components and extracellular matrix (ECM). Advanced glycation end-products (AGEs), the final derivative of the Maillard or browning reaction, are known to accumulate in musculoskeletal tissues in old age and are thought to play a role in the development of sarcopenia. AGEs preferentially accrue on the long-lived extracellular matrix (ECM) proteins, especially collagens, since their formation relies on its precursors' stochastic reaction (glucose and proteins) via the Maillard reaction. In addition to having a long half-life, collagens are rich in repeating arginine and lysine amino acids that potentiate the reaction between collagen and AGE precursors, further predisposing collagen to these glycation cross-links. Non-enzymatic cross-linking by AGEs decrease collagen's susceptibility to degradation by matrix metalloproteinases, causing the build-up of collagen and subsequent stiffening of the usually pliant skeletal muscle ECM.

Skeletal muscle tissue is a densely packed tissue that is relatively difficult to decellularize and difficult to maintain extracellular matrix structure and composition without disrupting the matrix architecture or stripping out important components that regulate tissue regeneration. Thus, methods of decellularizing animal tissue, in particular, tissues having enhanced AGE crosslinks due to aging or disease, are needed in order to provide better models of disease.

### SUMMARY

Provided herein are methods for preserving the existing extracellular matrix in animal tissue using decellularization with zwitterionic detergents combined with methods to reduce AGE cross-links and reduce tissue stiffness which are often associated with aging and disease. Bioinks for three-dimensional (3D) printing may be prepared from healthy and diseased tissues in order to mimic those conditions of the microenvironment.

An aspect of the disclosure provides a method of decellularizing an animal tissue comprising digesting the animal tissue; treating the animal tissue with a surfactant; treating the animal tissue with at least one zwitterionic detergent to form a decellularized animal tissue; and treating the decellularized animal tissue with at least one AGE inhibitor to reduce AGE crosslinking in the decellularized animal tissue.

In some embodiments, the animal tissue is skeletal muscle tissue. In some embodiments, the animal tissue is a diseased tissue having enhanced AGE crosslinking in an extracellular matrix as compared to healthy animal tissue. In some embodiments, the enhanced AGE crosslinking is caused by aging, diabetes, muscular dystrophy, disuse atrophy, denervation atrophy, trauma, polymyositis, or amyotrophic lateral sclerosis.

In some embodiments, the surfactant is selected from the group consisting of t-Octylphenoxypolyethoxyethanol, polysorbate 20, and polysorbate 80. In some embodiments, the at least one zwitterionic detergent comprises 3-[(3-cholamidopropyl)dimethylammonio]-1-propanesulfonate (CHAPS). In some embodiments, the at least one zwitterionic detergent comprises sulfobetaine-16 (SB-16). In some embodiments, the at least one zwitterionic detergent comprises CHAPS and SB-16. In some embodiments, the at least one AGE inhibitor is selected from the group consisting of alargebrium chloride 711 (ALT-711), ALT-946, ALT-462, ALT-486, ALT-TRC4186, and aminoguanidine. In some embodiments, the at least one AGE inhibitor is ALT-711. In some embodiments, the decellularized animal tissue is treated with 0.25-30% (w/v) of ALT-711.

Another aspect of the disclosure provides a method of manufacturing a bioink composition comprising the preparation of a decellularized animal tissue as described herein; grinding the decellularized animal tissue to form decellularized tissue matrix material powder, and dissolving the powder in a digest solution to form the bioink composition. In some embodiments, the grinding step is performed at cryotemperatures in the range of—(150-200)° C. In some embodiments, the digest solution comprises pepsin.

Another aspect of the disclosure provides a method of manufacturing an in vitro model of an animal tissue comprising manufacturing a bioink composition as described herein; and printing the bioink composition to form the in vitro model of the animal tissue. In some embodiments, the animal tissue is a healthy tissue. In some embodiments, the animal tissue is a diseased tissue having enhanced AGE crosslinking in an extracellular matrix as compared to healthy animal tissue. In some embodiments, the enhanced AGE crosslinking is caused by aging, diabetes, muscular dystrophy, disuse atrophy, denervation atrophy, trauma, polymyositis, or amyotrophic lateral sclerosis.

Another aspect of the disclosure provides a bioink for physiological 3D-printing, wherein the bioink is made by a method as described herein.

### BRIEF DESCRIPTION OF THE DRAWINGS

FIGS. 1A-C. Three views (A-C) of the gross morphology of rat gastrocnemius after decellularization according to some embodiments of the disclosure.

FIG. 2. Picrosirius stained rat gastrocnemius after decellularization according to some embodiments of the disclosure.

FIG. 3. Mouse gastrocnemius treated with ALT-711 shows reduced levels of AGEs in the decellularized muscle matrix (DMM).

FIGS. 4A-B. Treatment of human DMM with ALT-711 reduced AGEs when normalized to protein (A) and hydroxyproline (B).

### DETAILED DESCRIPTION

Embodiments of the disclosure provide methods for decellularizing animal tissue which may be combined with methods for reducing AGE cross-links to reduce tissue stiffness which is often associated with aging and disease. Bioinks may be created from the healthy and diseased decellularized tissue in order to create 3D printed in vitro models mimicking the microenvironment of these conditions.

Animal tissue may be obtained from any human or non-human animal tissue that is suitable for decellularization. In some embodiments, the tissue is selected from cardiac, dermal, pulmonary, renal, intestinal, hepatic, pancreatic, tendon, ligament, artery, vein, diaphragm, cartilage, or skeletal muscle tissue. Exemplary non-human sources include, but are not limited to, monkeys, pigs, sheep, goats, cows, horses, dogs, cats, rabbits, guinea pigs, gerbils, hamsters, rats, mice, and/or other non-human mammals.

Decellularization is a process used to isolate the extracellular matrix (ECM) of a tissue from its inhabiting cells, leaving an ECM scaffold of the original tissue, which can be used in artificial organ and tissue regeneration. The acquired ECM scaffold can be used to reproduce a functional organ by introducing progenitor cells, or adult stem cells, and allowing them to differentiate within the scaffold to develop into the desired tissue. The produced organ or tissue can be transplanted into a patient.

As used herein, a “decellularized tissue” is any tissue from which most or all of the cells that are normally found growing in the extracellular matrix of the tissue have been removed, e.g., a tissue lacking at least about 80, 85, 90, 95, 99, 99.5, or 100% of the native cells (or any percentage in between).

The extent of decellularization can be determined histochemically, for example, by staining the tissue with hematoxylin and eosin using standard techniques.

Immunohistochemical staining can also be used, for example, to visualize cell specific markers such as Beta-actin and histocompatibility antigens—an absence of such markers being a further indication of decellularization. In certain aspects, immunohistochemical antibody staining can be used to identify specific immunogens associated with rejection (e.g., HLA-DR) and the removal of cellular DNA below the detection levels of current analysis methods.

In various aspects, the methods of the present disclosure yield tissue, wherein the tissue is characterized by a substantial absence of positive staining for cell nuclei. In various aspects, the methods of the present disclosure yield tissue, wherein the tissue is characterized by a substantial absence of cellular DNA. In various aspects, the methods of the present disclosure yield tissue, wherein the tissue is characterized by a substantial absence of immunogenic proteins. In various aspects, the methods of the present disclosure yield tissue, wherein the tissue is characterized by more than 70-80, 70, 80, 90, 95, or 99% reduction in cytoskeletal proteins levels. In further aspects, the cytoskeletal proteins are vimentin, beta-actin, alpha-actin, myosin, tubulin, desmin, and vinculin.

A decellularization method as described herein may include an initial step of digesting the animal tissue, e.g. using an enzyme such as trypsin. In some embodiments, the tissue is exposed to a digest enzyme at a concentration of about 0.02-0.5%, e.g., at about 0.02, 0.03, 0.04, 0.05, 0.06, 0.07, 0.08, 0.09, 0.1, 0.15, 0.2, 0.25, 0.3, 0.35, 0.4, 0.45, or 0.5% (or any percentage in between). In certain embodiments, the tissue is exposed to a digest enzyme for at least about 1-20 hours, e.g. about 1, 2, 3, 4, 5, 6, 7, 8, 9, 10, 11, 12, 13, 14, 15, 16, 17, 18, 19, 20 hours (or any time period in between). In some embodiments, the digested tissue is washed prior to the next step. Any physiologically compatible solution can be used for washing such as deionized or distilled water, phosphate buffered saline (PBS), or any other biocompatible saline solution.

The digested tissue may then be treated with one or more surfactants, e.g. anionic or cationic surfactants. Suitable surfactants include, but are not limited to, t-Octylphenoxypolyethoxyethanol (TRITON X-100™), polysorbate 20, polysorbate 80, sodium dodecyl sulfate, sodium deoxycholate, polyoxyethylene (20) sorbitan monolaurate, etc. In some embodiments, the decellularization solution comprises 0.05%-2%, e.g., 0.05%, 0.06%, 0.07%, 0.08%, 0.09%, 0.1%, 0.2%, 0.3%, 0.4%, 0.5%, or more (or any percentage in between) of surfactant. In some embodiments, the tissue is incubated in the surfactant solution at 0-10° C., e.g., 0, 1, 2, 3, 4, 5, 6, 7, 8, 9, or 10 degrees Celsius (or any temperature in between), and optionally, gentle shaking is applied at 10, 20, 30, 40, 50, 60, 70, 80, 90, 100, 110, 120, 130, 140, or 150 rpm (or any rpm in between). The incubation can be for 1-96 hours, e.g., 1, 2, 3, 4, 5, 6, 7, 8, 9, 10, 11, 12, 15, 20, 24, 36, 48, 60, 72, 84, or 96 hours (or any time period in between). In some embodiments, the tissue is washed with a physiologically compatible solution as described herein after treatment with a surfactant.

The tissue may then be treated with at least one zwitterionic detergent to further kill and remove cells. The polar head groups of zwitterionic detergents contain both negatively and positively charged atomic groups, therefore the overall charge is neutral. Suitable zwitterionic detergents but include are not limited to 3-[(3-cholamidopropyl)dimethylammonio]-1-propanesulfonate (CHAPS), sulfobetaine-16 (SB-16), and N,N-Dimethyl-n-dodecylamine-N-oxide (LDAO). In some embodiments, the tissue is treated with 0.1-2 mM SB-16, e.g. 0.1, 0.2, 0.3, 0.4, 0.5, 0.6, 0.7, 0.8, 0.9, 1, 1.5, or 2 mM (or any amount in between). In some embodiments, the tissue is treated with 60-80 mM CHAPS, e.g. 62, 64, 66, 68, 70, 72, 74, 76, 78 mM CHAPS (or any amount in between). In some embodiments, the tissue is treated first with SB-16 then CHAPS or vice versa. In some embodiments, the tissue is incubated with the zwitterionic detergent at 0-10° C., e.g., 0, 1, 2, 3, 4, 5, 6, 7, 8, 9, or 10 degrees Celsius (or any temperature in between), and optionally, gentle shaking is applied at 10, 20, 30, 40, 50, 60, 70, 80, 90, 100, 110, 120, 130, 140, or 150 rpm (or any rpm in between). In some embodiments, the tissue is treated with the zwitterionic detergent for 1-96 hours, e.g., 1, 2, 3, 4, 5, 6, 7, 8, 9, 10, 11, 12, 15, 20, 24, 36, 48, 60, 72, 84, or 96 hours (or any time period in between). In some embodiments, the tissue is washed with a physiologically compatible solution as described herein after treatment with a zwitterionic detergent.

In some embodiments, the decellularized tissue is treated with an endonuclease, e.g. a DNase or RNase solution, e.g. for 30 minutes to 3 hours or more to degrade cellular genetic materials. The tissue may also be sterilized with a disinfectant. In certain embodiments, the disinfectant is peracetic acid (PAA), for example at a concentration of 0.05, 0.1, 0.15, 0.2, 0.25, 0.3, 0.4, or 0.5% (or any percentage in between). In some embodiments, the tissue is washed with a physiologically compatible solution as described herein after treatment with DNase and/or a disinfectant.

In some embodiments, the decellularized tissue is treated with at least one AGE inhibitor to reduce AGE crosslinking in the decellularized tissue. Use of an AGE inhibitor allows for easier manipulatation of decellularized tissues, e.g. to create bioinks that have more effective bioink gelation. It also allows for processing of older tissues which are generally more available as compared to young tissue. Suitable AGE inhibitors include, but are not limited to, alargebrium chloride 711 (ALT-711), ALT-946, ALT-462, ALT-486, ALT-TRC4186, aminoguanidine and those described in U.S. Pat. No. 9,309,304 incorporated herein by reference. In some embodiments, the decellularized animal tissue is treated with 0.25-30% (w/v) of ALT-711. The tissue may be incubated with the AGE inhibitor at 30-45° C., e.g., 31, 32, 33, 34, 35, 36, 37, 38, 39, 40, 41, 42, 43, 44, or 45 degrees Celsius (or any temperature in between), and optionally, gentle shaking is applied at 10, 20, 30, 40, 50, 60, 70, 80, 90, 100, 110, 120, 130, 140, or 150 rpm (or any rpm in between). In some embodiments, the tissue is treated with the AGE inhibitor for 1-10 days, e.g., 1, 2, 3, 4, 5, 6, 7, 8, 9, or 10 days (or any time period in between). In some embodiments, the tissue is washed with a physiologically compatible solution after treatment with the AGE inhibitor.

In some embodiments, the decellularized tissue may be freeze-dried, e.g. at −80° C. Suitable lyophilization techniques are known in the art.

The decellularized tissue may be further processed for applications in tissue engineering, tissue allograft transplantation, regenerative medicine, and injectables using bioinks. Some embodiments provide a method of preparing a bioink useful for 3D-printing an in vitro model of the tissue. In some embodiments, further processing includes grinding the decellularized animal tissue to form decellularized tissue matrix material powder, e.g. via cryomilling, and dissolving the powder in a digest solution to form the bioink composition. In some embodiments, the grinding step is performed at—(150-200)° C. In some embodiments, the digest solution comprises pepsin. These bioinks retain the native chemicals in the extracellular matrix such as chemokines and growth factors that affect strategies aimed at tissue engineering and regenerative medicine.

Further embodiments provide a method of manufacturing an in vitro model of an animal tissue comprising manufacturing a bioink composition as described herein; and printing the bioink composition to form the in vitro model of the healthy or diseased animal tissue. The tissue may be a healthy tissue or a diseased tissue having enhanced AGE crosslinking in an extracellular matrix as compared to healthy animal tissue. In some embodiments, the enhanced AGE crosslinking is caused by aging, diabetes, muscular dystrophy, disuse atrophy, denervation atrophy, trauma, polymyositis, or amyotrophic lateral sclerosis.

Bioprinting includes the application of three-dimensional printing techniques for deposition of biological materials into desired patterns. Cell patterns are created layer-by-layer, such that cell function and viability can be preserved in the resulting printed construct and can be used for medical and/or tissue engineering purposes. In addition to ECM, the bioinks described herein may contain additional biological materials such as agarose, alginate, chitosan, collagen, gelatin, fibrin and hyaluronic acid. 3D bioprinting technologies that are compatible with the present disclosure include extrusion, inkjet, and laser assisted extrusion technologies. Extrusion bioprinting is performed by extruding bioink with the appropriate mechanical properties (viscosity, elasticity and shear de-viscosity) through a nozzle using mechanical (piston or screw driven) or pneumatic pressure. Extrusion-based bioprinting (eg, bioplot or fused deposition modeling) involves dispensing the sticky bioink through a nozzle or syringe. After printing, the structure can be solidified (e.g. gelled) layer by layer, either physically or chemically. Suitable 3D printers capable of performing the aforementioned techniques are known in the art.

Also described herein is a kit comprising one or more solutions as described herein. In some embodiments, the kit comprises a surfactant solution and a zwitterionic detergent solution as described herein. In some embodiments, the kit comprises an AGE inhibitor solution. In some embodiments, the kit comprises a cleaning and/or disinfectant solution. In some embodiments, the kit comprises a nuclease solution. In some embodiments, the kit includes instructions for use of the kit and its contents. In some embodiments, the kit includes a sterile container. In some embodiments, the kit includes labeling with directions for use. In some embodiments, the kit comprises instructions for contacting a tissue with one or more of the solutions described herein. In some embodiments, the kit further comprises instructions for performing a method described herein.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE

### Exemplary Decelluarization Method

i. Gastrocnemius is thawed in a 37° C. water bath and then butterflied.
ii. The muscles are washed in a T-25 flask (standing vertical) with 20 ml of deionized water per well 3 times for 10 minutes each.
iii. Tissue is then treated with 20 ml of 0.25% Trypsin for 12-16 hours in a T-25 flask (standing vertical) on an orbital shaker plate. Use tape to secure the flask in a vertical position.
iv. Trypsin treatment is followed by 3 20 ml 15 minute deionized water washes per well.
v. Tissue is treated with 20 mL of 0.1% TRITON X-100™ in a T-25 flask for 8 hours at 4° C. The volume is replaced with fresh TRITON X-100™ and the tissue is treated for another 16 hours. Treatment is followed by 3 20 ml 15 minute deionized water washes per well.
vi. Samples are placed in 20 mL of 60 mM sodium deoxycholate with 0.6 mM SB-16 for 8 hours in a T-25 flask at 4° C. The volume is replaced with fresh sodium deoxycholate and SB-16 and the tissue is treated for another 16 hours.
vii. Samples are washed as described in deionized water and placed in 20 mL of 70 mM CHAPS for 8 hours in a T-25 mL flask at 4° C. The volume is replaced with fresh CHAPS and the tissue is treated for another 16 hours.
viii. Lastly, the tissue samples are washed with dI H20 as described previously and then treated with 5 ml of 1,200 kU/mL DNase for 1 hour, washed 3 times with deionized water, and then sterilized with 0.1% Peracetic acid for 24 hours.
ix. Samples are washed 5 times for 15 minutes each in 20 mL sterile 1×PBS.
x. Decellularized muscle is then frozen at −80° C. and lyophilized until dry.

### Results

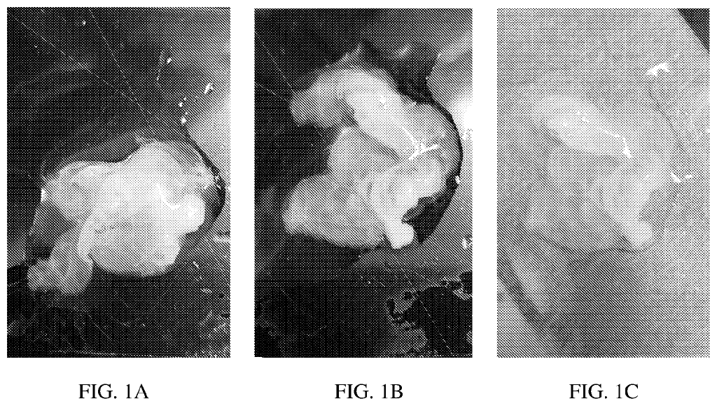

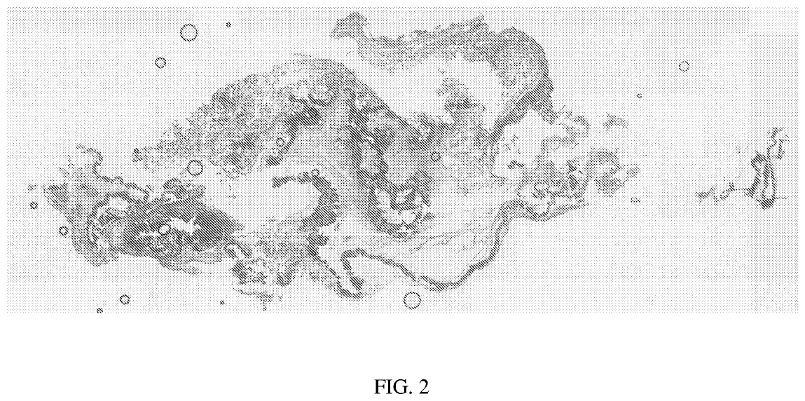

We show through gross morphology (FIGS. 1(A-C) and picrosirius red histological staining (FIG. 2) that the above method is adequate to remove all of the cellular material, leaving a scaffold rich in collagenous material.

Exemplary method for reducing AGEs in decellularized muscle xi. 10 mM ALT-711 is added to each well, and then placed in the 37° C. incubator for 5 days.

xii. After 5 days, the samples are washed 3 times with diH20 each, and then the samples are frozen at −80° C.

### Results

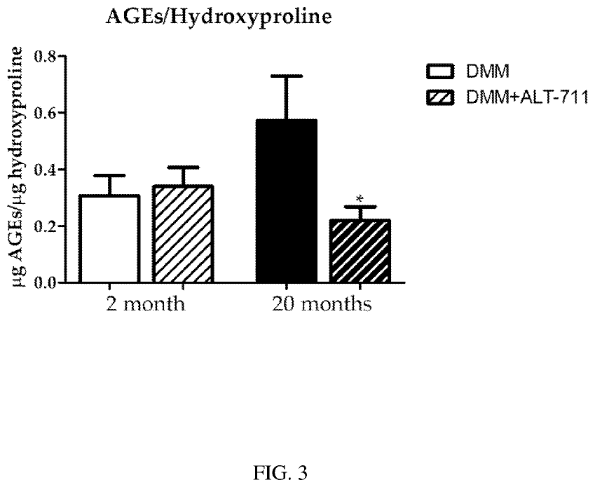

We applied this process to both 2-month (young adult) and 20-month (aged adult) C57BL6 mouse gastrocnemius and show that treating with ALT-711 reduces the higher level of AGEs seen in the 20-month decellularized muscle matrix (DMM) to young adult levels (FIG. 3).

In addition, human decellularized muscle from MTF Biologics was also tested for feasibility to reduce AGEs in older human samples using the same protocol as described above.

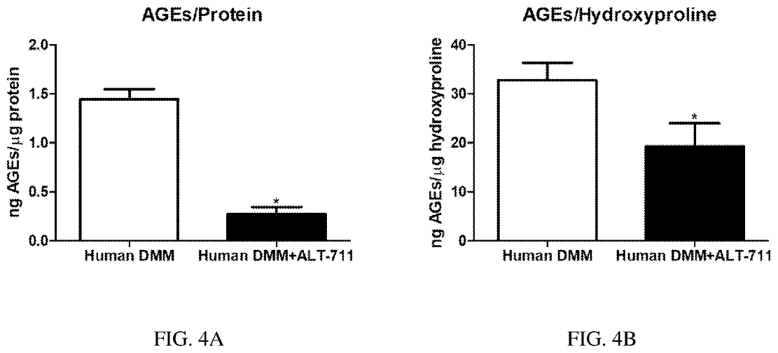

We treated human DMM that was created with an FDA approved decellularization protocol with ALT-711 and show that ALT-711 reduced AGEs when normalized to protein (FIG. 4A) and hydroxyproline (FIG. 4B).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of decellularizing an animal tissue comprising:
digesting the animal tissue;
treating the animal tissue with a surfactant;
treating the animal tissue with at least one zwitterionic detergent to form a decellularized animal tissue; and
treating the decellularized animal tissue with at least one advanced glycation end-product (AGE) inhibitor to reduce AGE crosslinking in the decellularized animal tissue.

2. The method of claim 1, wherein the animal tissue is skeletal muscle tissue.

3. The method of claim 1, wherein the animal tissue is a diseased tissue having enhanced AGE crosslinking in an extracellular matrix as compared to healthy animal tissue.

4. The method of claim 3, wherein the enhanced AGE crosslinking is caused by aging, diabetes, muscular dystrophy, disuse atrophy, denervation atrophy, trauma, polymyositis, or amyotrophic lateral sclerosis.

5. The method of claim 1, wherein the surfactant is selected from the group consisting of t-Octylphenoxypolyethoxyethanol, polysorbate 20, and polysorbate 80.

6. The method of claim 1, wherein the at least one zwitterionic detergent comprises 3-[(3-cholamidopropyl)dimethylammonio]-1-propanesulfonate (CHAPS).

7. The method of claim 1, wherein the at least one zwitterionic detergent comprises sulfobetaine-16 (SB-16).

8. The method of claim 1, wherein the at least one zwitterionic detergent comprises CHAPS and SB-16.

9. The method of claim 1, wherein the at least one AGE inhibitor is selected from the group consisting of alargebrium chloride 711 (ALT-711), ALT-946, ALT-462, ALT-486, ALT-TRC4186, and aminoguanidine.

10. The method of claim 1, wherein the at least one AGE inhibitor is ALT-711.

11. The method of claim 10, wherein the decellularized animal tissue is treated with 0.25-30% w/v of ALT-711.

12. The method of claim 1, wherein the animal tissue is human tissue.

13. A method of manufacturing a bioink composition comprising:
digesting an animal tissue;
treating the animal tissue with a surfactant;
treating the animal tissue with at least one zwitterionic detergent to form a decellularized animal tissue;
treating the decellularized animal tissue with at least one advanced glycation end-product (AGE) inhibitor to reduce AGE crosslinking in the decellularized animal tissue;
grinding the decellularized animal tissue to form decellularized tissue matrix material powder, and
dissolving the powder in a digest solution to form the bioink composition.

14. The method of claim 13, wherein the grinding step is performed at—(150-200)° C.

15. The method of claim 13, wherein the digest solution comprises pepsin.

16. A method of manufacturing an in vitro model of an animal tissue comprising:
manufacturing a bioink composition according to claim 13; and
printing the bioink composition to form the in vitro model of the animal tissue.

17. The method of claim 16, wherein the animal tissue is a healthy tissue.

18. The method of claim 16, wherein the animal tissue is a diseased tissue having enhanced AGE crosslinking in an extracellular matrix as compared to healthy animal tissue.

19. The method of claim 18, wherein the enhanced AGE crosslinking is caused by aging, diabetes, muscular dystrophy, disuse atrophy, denervation atrophy, trauma, polymyositis, or amyotrophic lateral sclerosis

20. A bioink for physiological 3D-printing, wherein the bioink is made by the method of claim 13.
